(19) United States
(12) Patent Application Publication
(10) Pub. No.: US 2008/0021466 A1
(43) Pub. Date: Jan. 24, 2008

(54) SPINE TREATMENT DEVICES AND METHODS

(76) Inventors: John H. Shadduck, Tiburon, CA (US); Csaba Truckai, Saratoga, CA (US); Robert Luzzi, Pleasanton, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/781,051

(22) Filed: Jul. 20, 2007

Related U.S. Application Data
(60) Provisional application No. 60/831,915, filed on Jul. 20, 2006.

(51) Int. Cl. A61B 17/58; A61B 17/08; A61F 2/44; A61B 17/56

(52) U.S. Cl. 606/61; 606/100; 606/151; 623/17.13

(57) ABSTRACT

A modular implant device and method for dynamic stabilization of a spine segment that can be implanted in a minimally invasive posterior approach. The implant device has superior and inferior body portions configured for engaging two spaced apart spinous processes to limit extension and, optionally, flexion while off-loading a spine segment. The implant device can stabilize a spine segment, re-distribute loads with the spine segment and still allow spine lateral bending and torsion. Implantation of the device is reversible and adjustable post-implantation.

### CROSS-REFERENCE TO RELATED APPLICATIONS

This application claims the benefit of U.S. Provisional Patent Application No. 60/831,915 filed Jul. 20, 2006, the entire contents of which are incorporated herein by reference and should be considered a part of this specification.

### BACKGROUND OF THE INVENTION

1. Field of the Invention

The invention relates generally to implant systems and methods for treating a spine disorder, and more particularly relates to minimally invasive implant devices configured for engaging spaced apart spinous processes to off-load the intermediate motion segment for re-distributing loads within a spine segment while still allowing for flexion, extension, lateral bending and torsion.

2. Description of the Related Art

Thoracic and lumbar spinal disorders and discogenic pain are major socio-economic concerns in the United States affecting over 70% of the population at some point in life. Low back pain is the most common musculoskeletal complaint requiring medical attention; it is the fifth most common reason for all physician visits. The annual prevalence of low back pain ranges from 15% to 45% and is the most common activity-limiting disorder in persons under the age of 45.

Degenerative changes in the intervertebral disc often play a role in the etiology of low back pain. Many surgical and non-surgical treatments exist for patients with degenerative disc disease (DDD), but often the outcome and efficacy of these treatments are uncertain. In current practice, when a patient has intractable back pain, the physician's first approach is conservative treatment with the use of pain killing pharmacological agents, bed rest and limiting spinal segment motion. Only after an extended period of conservative treatment will the physician consider a surgical solution, which often is spinal fusion of the painful vertebral motion segment. Fusion procedures are highly invasive procedure that carries surgical risk as well as the risk of transition syndrome described above wherein adjacent levels will be at increased risk for facet and discogenic pain.

More than 150,000 lumbar and nearly 200,000 cervical spinal fusions are performed each year to treat common spinal conditions such as degenerative disc disease and spondylolisthesis, or misaligned vertebrae. Some 28 percent are multi-level, meaning that two or three vertebrae are fused. Such fusions “weld” unstable vertebrae together to eliminate pain caused by their movement. While there have been significant advances in spinal fusion devices and surgical techniques, the procedure does not always work reliably. In one survey, the average clinical success rate for pain reduction was about 75%; and long time intervals were required for healing and recuperation (3-24 months, average 15 months). Probably the most significant drawback of spinal fusion is termed the “transition syndrome” which describes the premature degeneration of discs at adjacent levels of the spine. This is certainly the most vexing problem facing relatively young patients when considering spinal fusion surgery.

Many spine experts consider the facet joints to be the most common source of spinal pain. Each vertebra possesses two sets of facet joints, one set for articulating to the vertebra above and one set for the articulation to the vertebra below. In association with the intervertebral discs, the facet joints allow for movement between the vertebrae of the spine. The facet joints are under a constant load from the weight of the body and are involved in guiding general motion and preventing extreme motions in the trunk. Repetitive or excessive trunkal motions, especially in rotation or extension, can irritate and injury facet joints or their encasing fibers. Also, abnormal spinal biomechanics and bad posture can significantly increase stresses and thus accelerate wear and tear on the facet joints.

Recently, technologies have been proposed or developed for disc replacement that may replace, in part, the role of spinal fusion. The principal advantage proposed by complete artificial discs is that vertebral motion segments will retain some degree of motion at the disc space that otherwise would be immobilized in more conventional spinal fusion techniques. Artificial facet joints are also being developed. Many of these technologies are in clinical trials. However, such disc replacement procedures are still highly invasive procedures, which require an anterior surgical approach through the abdomen.

Clinical stability in the spine can be defined as the ability of the spine under physiologic loads to limit patterns of displacement so as to not damage or irritate the spinal cord or nerve roots. In addition, such clinical stability will prevent incapacitating deformities or pain due to later spine structural changes. Any disruption of the components that stabilized a vertebral segment (i.e., disc, facets, ligaments) decreases the clinical stability of the spine.

Improved devices and methods are needed for treating dysfunctional intervertebral discs and facet joints to provide clinical stability, in particular: (i) implantable devices that can be introduced to offset vertebral loading to treat disc degenerative disease and facets through least invasive procedures; (ii) implants and systems that can restore disc height and foraminal spacing; and (iii) implants and systems that can re-distribute loads in spine flexion, extension, lateral bending and torsion.

### SUMMARY OF THE INVENTION

In accordance with one embodiment, a spine implant device is provided. The spine implant device comprises an implant body comprising a superior end portion with a first contact surface configured for contacting a first spinous process, and an inferior end portion with a second contact surface configured for contacting a non-adjacent second spinous process to off-load vertebral motion segments intermediate the first and second spinous processes.

In accordance with another embodiment, a spine implant device is provided comprising a biocompatible implant body. The implant body comprises a superior end portion and an inferior end portion, the end portions each having a U-shaped structure configured for grippable engagement of a spinous process, and a resilient medial portion between the superior and inferior end portions, the superior and inferior end portions spaced apart such that the U-shaped structures grippably engage non-adjacent spinous processes.

In accordance with still another embodiment, a spine implant device is provided comprising an implant body. The implant body comprises a superior end portion and an inferior end portion, the end portions each comprising at least one saddle portion configured to contact a spinous process, and a medial portion between the superior and inferior end portions, the superior and inferior end portions spaced apart such that the saddle portions engage non-adjacent spinous processes.

In accordance with yet another embodiment, a method of treating an abnormal spine segment of a patient is provided. The method comprises implanting a stabilization device such that a first superior end of the device engages a first spinous process, and such that a second inferior end of the device engages a second non-adjacent spinous process to thereby off-load at least one level of intervertebral discs and facet joints between said first and second engaged spinous processes.

### BRIEF DESCRIPTION OF THE DRAWINGS

The features and advantages of the embodiments discloses herein, and the manner of attaining them, will become apparent by reference to the following description of preferred embodiments taken in conjunction with the accompanying drawings, wherein:

FIG. 1 is a schematic perspective of an implant device for engaging spaced apart spinous processes.

FIG. 2 is a schematic view of an alternative device similar to that of FIG. 1.

FIG. 3 is a schematic view of the spine implant device of FIG. 2 implanted in a spine segment.

FIG. 4 is a schematic view of a plurality of implant devices of FIG. 2 implanted in a spine segment.

FIG. 5 is a schematic view of another embodiment of an implant device.

FIG. 6 is a schematic view of a plurality of implant devices of FIG. 2 implanted in different levels in a spine.

FIG. 7 is a schematic perspective view of an alternative implant device for engaging non-adjacent spinous processes.

FIG. 8 is a schematic view of the implant device of FIG. 7 implanted in a spine engaging non-adjacent spinous processes.

FIG. 9 is a schematic view of an alternative implant device similar to that of FIGS. 7-8.

FIGS. 10A and 10B are schematic views of an alternative implant device for engaging spaced apart spinous processes.

FIG. 11 is a schematic view of the implant device of FIGS. 10A-10B implanted in a spine and engaging non-adjacent spinous processes.

FIG. 12 is a schematic view of an alternative implant device similar to that of FIGS. 10A-10B.

FIG. 13 is a schematic view of the implant device of FIG. 12 implanted in a spine and engaging non-adjacent spinous processes.

FIG. 14 is a schematic view of an alternative implant device similar to that of FIG. 13.

### DETAILED DESCRIPTION OF THE PREFERRED EMBODIMENT

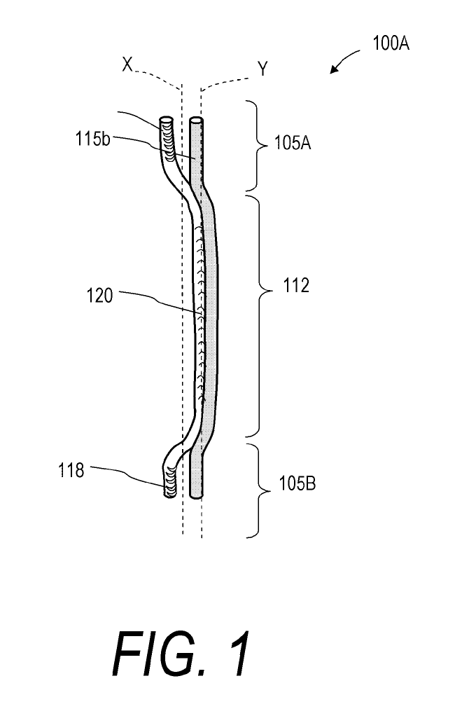

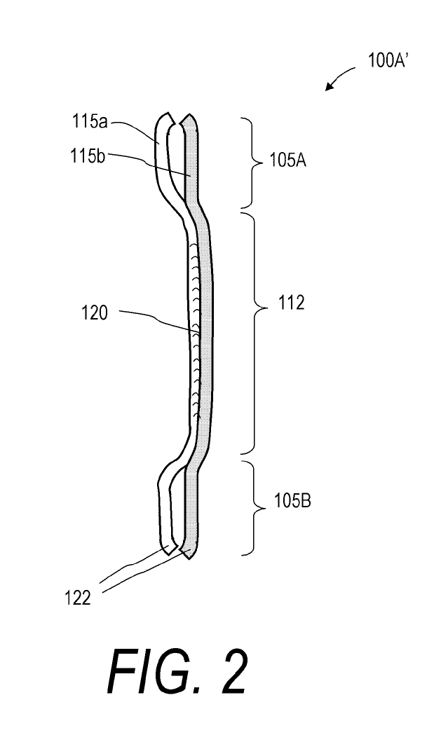

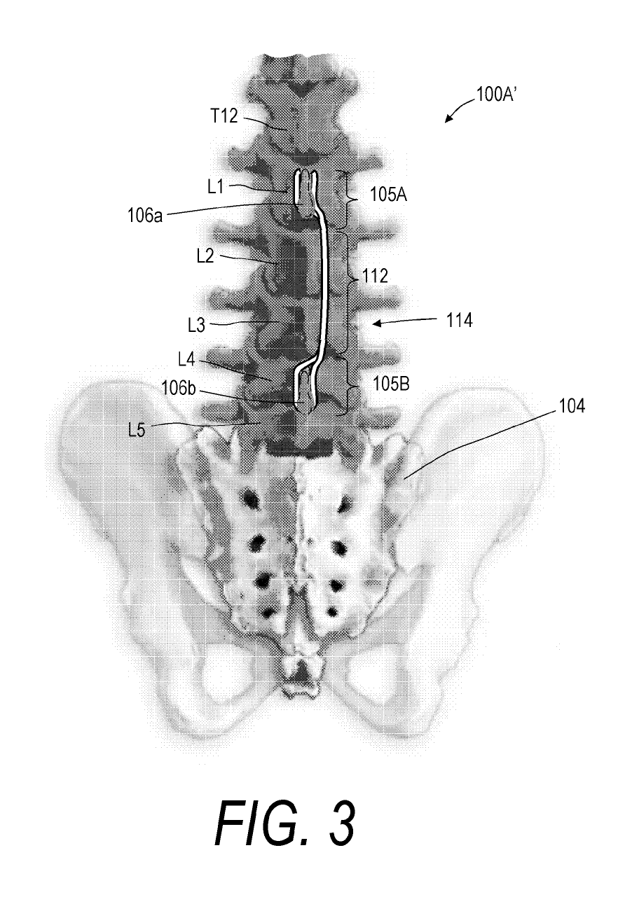

FIGS. 1 and 2 illustrate an embodiment of a spine implant device 100A having a first end portion or superior end 105A and a second end portion or inferior end 105B that can engage opposing surfaces of spaced apart spinous processes 106a and 106b (FIG. 3). In FIG. 1, a medial portion 112 of the implant that extends between the superior and inferior ends 105A, 105B can extend any suitable dimension for engaging spinous processes that are spaced apart by one or more intermediate vertebral bodies 114. In FIG. 1, the device provides first and second elements 115a and 115b that can engage the laterally outward surfaces of the spinous processes. In one embodiment, the first and second elements 115a, 115b can be welded together. In the illustrated embodiments, the first and second elements 115a, 115b define a slot at the superior end 105A and a slot at the inferior end 105B, said slots sized to receive the spinous processes 106a, 106b of nonadjacent vertebrae therein. In one embodiment, the first and second elements 115a and 115b can have gripping surfaces, such as a rasp or barbs that can engage the spinous processes 106a, 106b. The implant device 100A can have a body 120 made of the first and second elements 115a, 115b, which can be wire elements. In another embodiment, the body 120 can be a unitary body. The body 120 can be made of, for example, a plastic or metal material, such as a shape memory alloy. However, any material suitable for use in spinal implants can also be used. In the illustrated embodiment, the body 120 extends along a longitudinal axis Y that is offset from the longitudinal axis X passing through the slots and would generally correspond to the longitudinal axis through the spinous processes.

FIG. 2 depicts another embodiment of a spine implant device 100A′. The implant device 100A′ is similar to the implant device 100A discussed above. Thus, the reference numerals used to designate corresponding components in the implant 100A′ and the implant 100A are identical. The first and second elements 115a and 115b of the implant device 100A′ further include tip portions 122 at the superior and inferior ends 105A, 105B that can be resilient and can extend around and further grip the spinous processes, such as the spinous processes 106a, 106b.

FIGS. 3, 4 and 6 are schematic views of a plurality of implant devices of FIG. 2 implanted in a spine segment to engage spaced apart spinous processes. FIG. 3 illustrates the implant 100A′ implanted in a spine segment, such that the superior end 105A of the implant 100A′ engages the spinous process 106a and the inferior end 105B of the implant 100A′ engages the spinous process 106b. In the illustrated embodiment, the spine segment is defined by the L1-L4 vertebrae above the sacrum 104. However, the implant device 100A′ can be implanted in other areas of the spine, such as in thoracic vertebrae.

The implant device 100A′ off-loads a spine segment between the spinous processes 106a, 106b to alleviate a load on the discs and facet joints of the spine segment. For example, the medial portion 112 is preferably resilient and can act like a spring to receive at least a portion of a load place on the spinal segment, thereby reducing the load placed on the discs and facet joints of the vertebrae that make up the spine segment. As discussed above, the superior and inferior ends 105A, 105B have tip portions 122 that can resiliently deflect to allow insertion of the corresponding spinous processes 106a, 106b into the slots defined at the superior and inferior ends 105A, 105B. The medial portion 112 extends along an axis generally parallel to an axis through the spinous processes 106a, 106b.

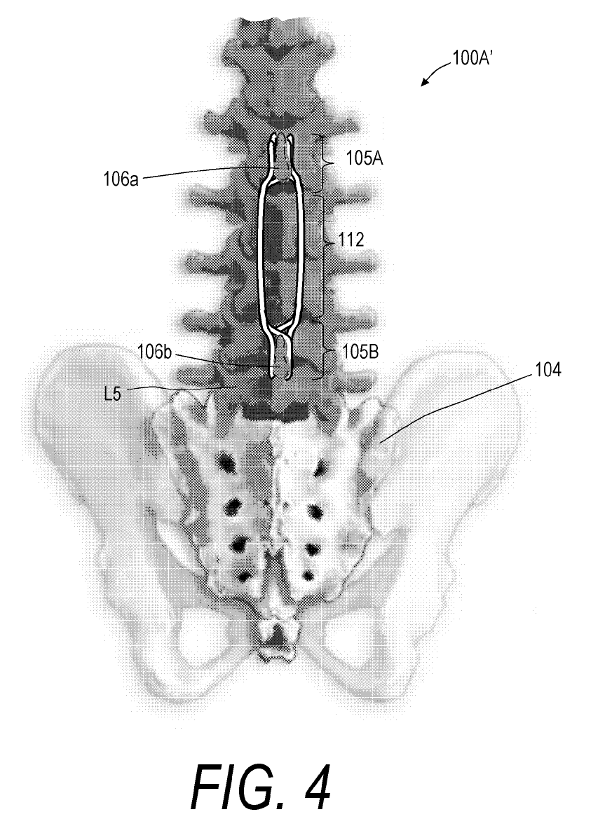

FIG. 4 illustrates two implant devices 100A′ implanted on the spine segment such that the superior ends 105A of the devices 100A′ engage the spinous process 106a and the inferior ends 105B engage the spinous process 106b. As shown in FIG. 4, the two implants 100A′ are implanted such that the medial portions 112 of the implants 100A′ extend on opposite sides of the spinous processes 106a, 106b. Though the illustrated embodiment shows two implant devices 100A′ coupled to the spinous processes 106a, 106b, one of ordinary skill in the art will recognize that more than two implant devices 100A′ can be coupled to the spinous processes 106a, 106b.

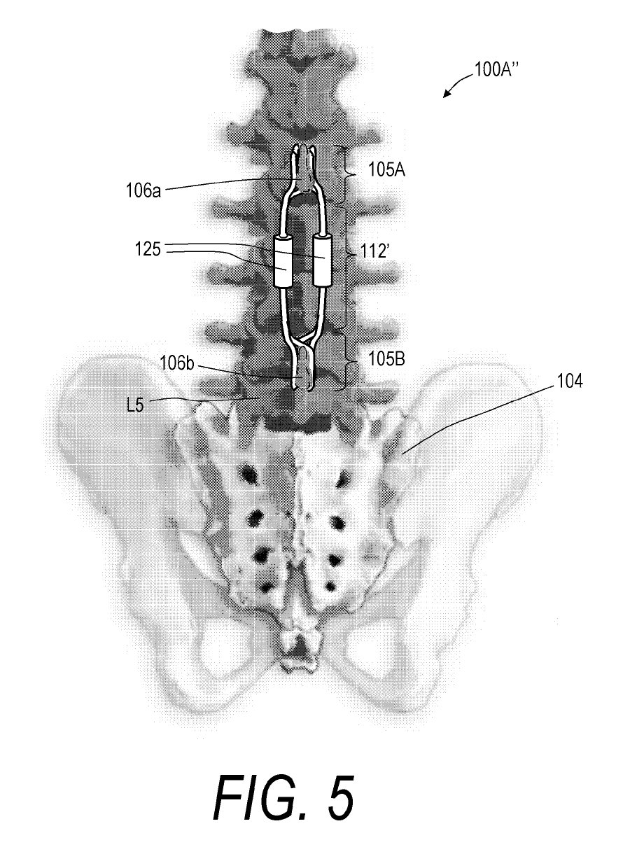

FIG. 5 depicts another embodiment of an implant device 100A″. The implant device 100A″ is similar to the implant device 100A′ discussed above. Thus, the reference numerals used to designate corresponding components in the implant 100A″ and the implant 100A′ are identical. In the illustrated embodiment, two implant devices 100A″ are shown, each having a medial portion 112′ incorporating a length-adjustment mechanism 125. The length-adjustment mechanism 125 can be a threaded turnbuckle mechanism, set screws or the like and can be actuated to adjust the length of the implant device 100A″ by adjusting the length of the medial portion 112.

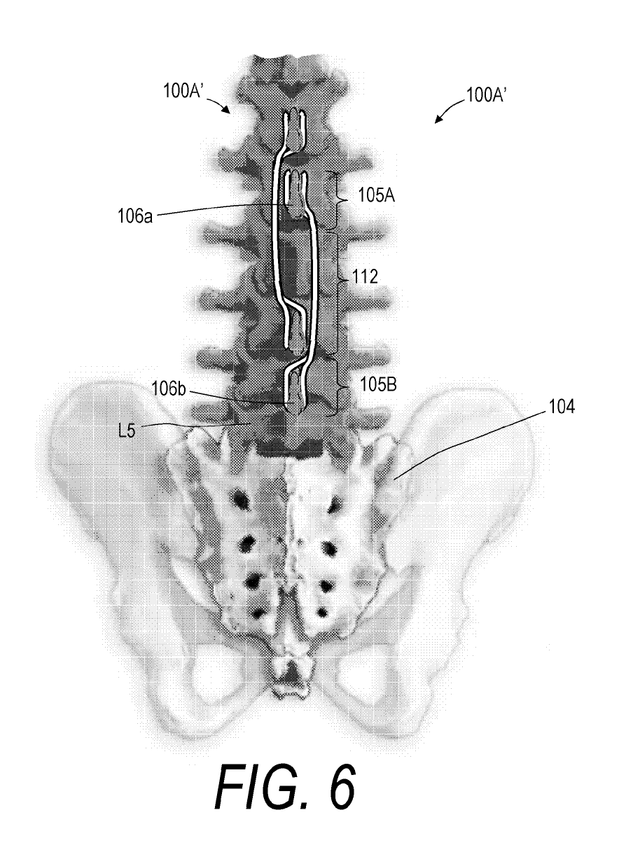

FIG. 6 shows a plurality of implant devices 100A′, such as those shown in FIG. 2, but wherein each implant device 100A′ is implanted at a different spinal level. In the illustrated embodiment, one implant device 100A′ is coupled to a first pair of spinous processes 106a, 106b, and a second implant device 100A′ is coupled to a second pair of spinous processes 106c, 106d.

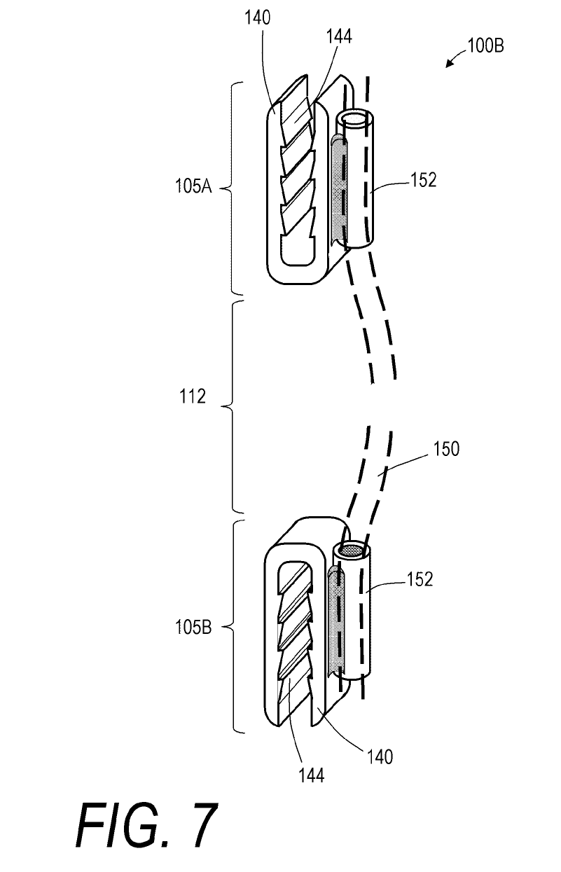

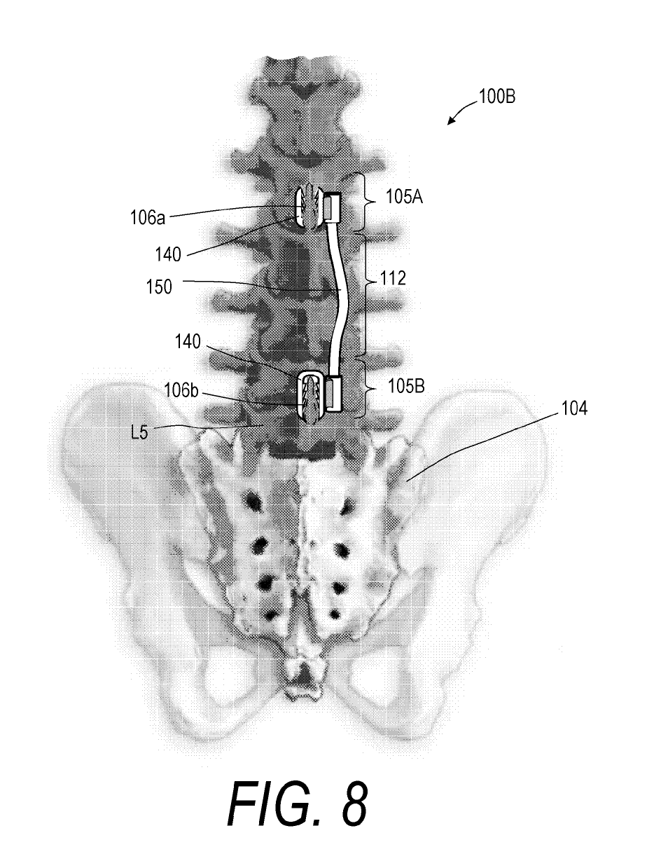

FIG. 7 depicts another embodiment of an implant device 100B having a superior end portion 105A and inferior end portion 105B that can each engage spaced apart spinous processes 106a and 106b (FIG. 8). The implant device 100B includes U-shaped members 140 with gripping features 144 for pressing over and grippingly engaging a spinous process. The U-shaped members 140 can be, for example, of a metal or polymer material. In FIG. 7, the medial portion 112 of the implant can extend any suitable dimension for engaging the targeted spinous processes and can comprise an extension member 150 (shown in phantom) that is lockable in sleeve portions 152 using any suitable locking mechanism. In one embodiment, the extension member 150 can be press-fit into the sleeve portions 152. In another embodiment, the extension member 150 can be lockingly coupled to the sleeve portions 152 via an adhesive or a set screw.

In FIG. 7, the gripping features 144 can be barbs, rasp features, teeth and the like. Further, the gripping features 144 can be biocompatible tissue adhesives such as cyanoacrylate that can be injected through the U-shaped body 140 from an external source (e.g., a syringe) that is connected to a port (not shown) in the body 144. In another embodiment, the U-shaped body 140 can be coupled to an electrical source for causing electrically or thermally induced sacrifice of surfaces of a reservoir to release the adhesive. In another embodiment, the U-shaped body 140 can have a gripping structure 144 that comprises an osmotic polymer or a natural material such as seaweed for expansion and gripping the engaged spinous process.

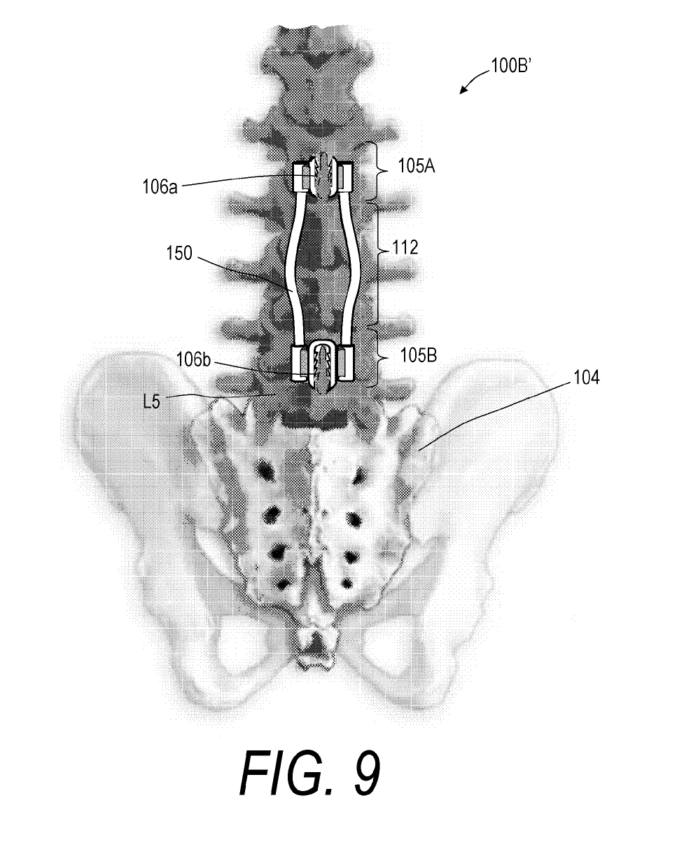

FIG. 9 illustrates an implant device 100B′, which is similar to the implant device 100B in FIG. 7. The implant device 100B′ is similar to the implant device 100B discussed above. Thus, the reference numerals used to designate corresponding components in the implant 100B′ and the implant 100B are identical. In the illustrated embodiment, the implant device 100B′ has first and second bi-lateral extension members 150, which can be rods, extending between the superior and inferior ends 105A, 105B of the implant device 100B′.

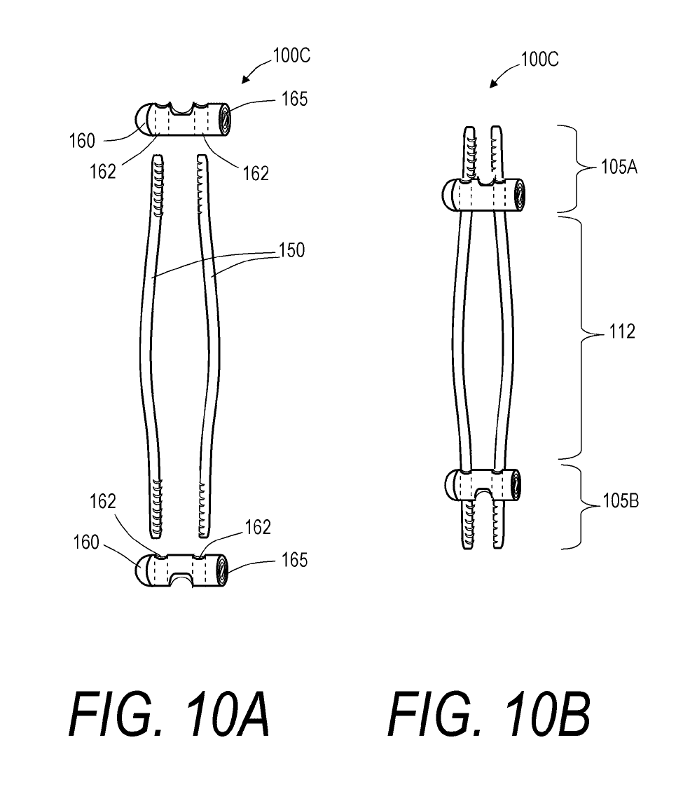

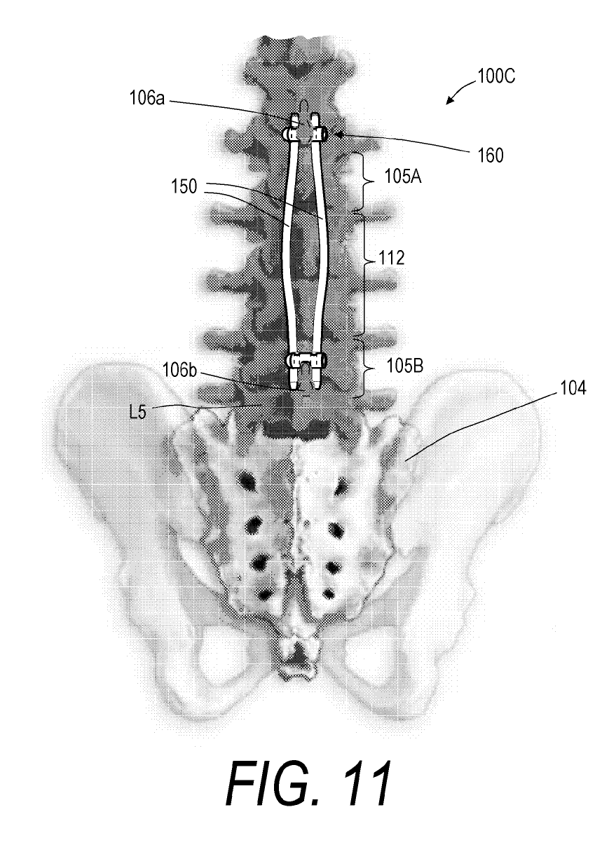

FIGS. 10A-10B illustrate another embodiment of an implant device 100C having a superior end portion 105A and inferior end portion 105B that each can engage spaced apart spinous processes 106a and 106b (FIG. 11). As shown in FIGS. 10A-10B, the implant device 100C can be assembled in situ from rods 150 and saddle members 160 that have bores 162 therein for receiving the rods 150. The rods 150 can have gripping surfaces, such as rasps or barbs to engage the spinous processes 106a, 106b. Set screws 165 can be used to lock the rods 150 together. However, other suitable locking mechanisms can be used. The rods 150 and saddle members 160 can be made of a metal or polymer material. However, any suitable material for use in spinal implants can be used.

In FIG. 11, the implant device 100C of FIGS. 10A-10B is implanted in a spine with the saddle members 160 engaging non-adjacent spinous processes 106a, 106b.

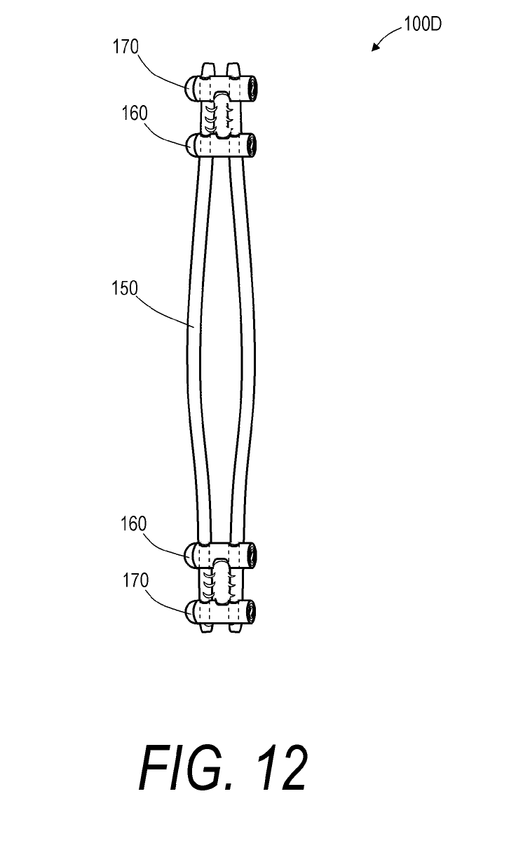

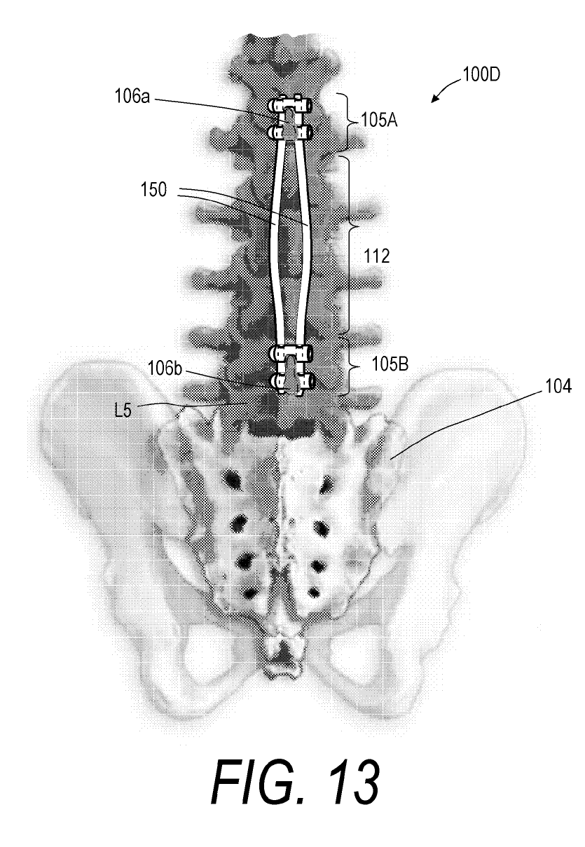

FIGS. 12 and 13 illustrate another embodiment of an implant device 100D that is similar to that of FIGS. 10A-11 except that it includes additional saddle members 170 for contacting opposing sides of each engaged spinous process. Accordingly, the spinous process 106a is engaged by the saddle members 160, 170 at the superior end 105A of the implant 100D, and the spinous process 106b is engaged by the saddle members 160, 170 at the inferior end 105B of the implant 100D.

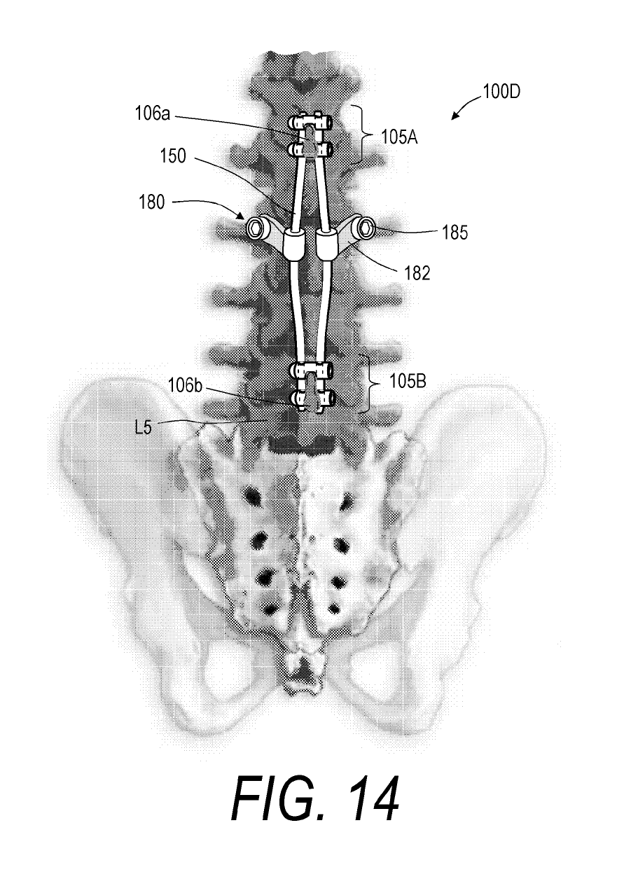

FIG. 14 illustrates another embodiment of an implant device and system that can includes the implant embodiments of FIG. 8, 9, 11 or 13 together with an intermediate component 180 that can receives the rod portions 150 of an implant either in a fixed manner or a slidable manner for controlling flexing of the rods 150. The intermediate component 180 can have bi-lateral bracket portions 182 that can be fixed in pedicles on either side of the spinous process by screws 185, as in known in the art. The intermediate component 180 can be made of a metal or polymer material, but can be made of any suitable material used in spinal implants. Preferably, the bracket portions 182 of the intermediate component 180 are made of a material suitable for controlling the flexing of the rods 150.

Certain embodiments described above provide new ranges of minimally invasive, reversible treatments that form a new category between traditional conservative therapies and the more invasive surgeries, such as fusion procedures or disc replacement procedures.

Certain embodiments include implant systems that can be implanted in a very minimally invasive procedure, and require only small bilateral incisions in a posterior approach. A posterior approach is highly advantageous for patient recovery. In some embodiment, the implant systems are “modular” in that separate implant components are used that can be implanted in a single surgery or in sequential surgical interventions. Certain embodiments of the inventive procedures are for the first time reversible, unlike fusion and disc replacement procedures. Additionally, embodiments include implant systems that can be partly or entirely removable. Further, in one embodiment, the system allows for in-situ adjustment requiring, for example, a needle-like penetration to access the implant.

In certain embodiments, the implant system can be considered for use far in advance of more invasive fusion or disc replacement procedures. In certain embodiments, the inventive system allows for dynamic stabilization of a spine segment in a manner that is comparable to complete disc replacement. Embodiments of the implant system are configured to improve on disc replacement in that it can augment vertebral spacing (e.g., disc height) and foraminal spacing at the same time as controllably reducing loads on facet joints—which complete disc replacement may not address. Certain embodiments of the implant systems are based on principles of a native spine segment by creating stability with a tripod load receiving arrangement. The implant arrangement thus supplements the spine's natural tripod load-bearing system (e.g., disc and two facet joints) and can re-distribute loads with the spine segment in spine torsion, extension, lateral bending and flexion.

Of particular interest, since the embodiments of implant systems are far less invasive than artificial discs and the like, the systems likely will allow for a rapid regulatory approval path when compared to the more invasive artificial disc procedures.

Other implant systems and methods within the spirit and scope of the invention can be used to increase intervertebral spacing, increase the volume of the spinal canal and off-load the facet joints to thereby reduce compression on nerves and vessels to alleviate pain associated therewith.

Although these inventions have been disclosed in the context of a certain preferred embodiments and examples, it will be understood by those skilled in the art that the present inventions extend beyond the specifically disclosed embodiments to other alternative embodiments and/or uses of the inventions and obvious modifications and equivalents thereof. For example, any of the implants disclosed above can be made of a metal material, polymer material, or shape memory alloy. In addition, while a number of variations of the inventions have been shown and described in detail, other modifications, which are within the scope of the inventions, will be readily apparent to those of skill in the art based upon this disclosure. It is also contemplated that various combinations or subcombinations of the specific features and aspects of the embodiments may be made and still fall within one or more of the inventions. Accordingly, it should be understood that various features and aspects of the disclosed embodiments can be combine with or substituted for one another in order to form varying modes of the disclosed inventions. Thus, it is intended that the scope of the present inventions herein disclosed should not be limited by the particular disclosed embodiments described above. Although particular embodiments of the present invention have been described above in detail, it will be understood that this description is merely for purposes of illustration. Specific features of the invention are shown in some drawings and not in others, and this is for convenience only and any feature may be combined with another in accordance with the invention. Further variations will be apparent to one skilled in the art in light of this disclosure and are intended to fall within the scope of the appended claims.

What is claimed is:

1. A spine implant device, comprising an implant body comprising a superior end portion with a first contact surface configured for contacting a first spinous process and an inferior end portion with a second contact surface configured for contacting a non-adjacent second spinous process to off-load vertebral motion segments intermediate the first and second spinous processes.

2. The spine implant device of claim 1, wherein the implant body comprises first and second elongated elements attached to each other, the elements defining the superior end portion and inferior end portion, the end portions configured to engage the first and second non-adjacent spinous processes.

3. The spine implant device of claim 1, wherein the first contact surface is configured for contacting an inferior surface of the first spinous process.

4. The spine implant device of claim 1, wherein the second contact surface is configured for contacting a superior surface of the second spinous process.

5. The spine implant device of claim 1, wherein at least one of the superior and inferior end portions comprise a shape memory alloy configured for engaging sides of the spinous process.

6. The spine implant device of claim 1, wherein the first and second contact surfaces are configured to grip the non-adjacent spinous processes.

7. The spine implant device of claim 1, wherein the implant body comprises a resilient medial body portion that extends between the superior and inferior end portions.

8. The spine implant device of claim 7, wherein the medial body portion is configured to extend along one side of the spinous processes of the spinal segment upon implantation of the implant device.

9. The spine implant device of claim 7, wherein the medial body portion is removably coupleable to the superior and inferior end portions.

10. The spine implant device of claim 7, wherein the medial body portion comprises a spring.

11. The spine implant device of claim 7, wherein the medial body portion includes length-adjustment mechanism configured to adjust the length of the medial body portion.

12. The spine implant device of claim 7, wherein the medial body portion comprises at least one rod.

13. The spine implant device of claim 1, wherein the implant body is configured so that the first and second contact surfaces contact non-adjacent spinous processes that are spaced apart by at least one intermediate vertebral body and spinous process.

14. A spine implant device, comprising:
a biocompatible implant body comprising
a superior end portion and an inferior end portion, the end portions each having a U-shaped structure configured for grippable engagement of a spinous process, and
a resilient medial portion between the superior and inferior end portions, the superior and inferior end portions spaced apart such that the U-shaped structures grippably engage non-adjacent spinous processes.

15. The spine implant device of claim 14, wherein an interior surfaces of the U-shaped structure includes surface features configured to fixably engage the spinous process.

16. The spine implant device of claim 15, wherein the surface features include at least one of teeth, barbs, projections, rasp-like features and biocompatible tissue adhesives.

17. The spine implant device of claim 14, wherein the medial body portion comprises at least one rod removably coupleable to the superior and inferior end portions.

18. A spine implant device, comprising:
an implant body comprising
a superior end portion and an inferior end portion, the end portions each comprising at least one saddle portion configured to contact a spinous process, and
a medial portion between the superior and inferior end portions, the superior and inferior end portions spaced apart such that the saddle portions engage non-adjacent spinous processes.

19. The spine implant device of claim 18, wherein the medial portion comprises at least one rod lockingly coupleable to the superior and inferior end portions, the rod configured to extend along one side of the spinous processes.

20. The spine implant device of claim 19, wherein the saddle portions are coupleable at one of a plurality of axial positions along the rod to vary the length of the medial body portion.

21. The spine implant device of claim 19, wherein the superior end portion comprises a first saddle portion configured to engage an inferior surface of a first spinous process and a second saddle portion configured to engage a superior surface of the first spinous process.

22. The spine implant device of claim 21, further comprising an intermediate component coupled to the rod and to a pedicle of a vertebra between the superior and inferior end portions, the intermediate component configured to control the flexure of the rod.

23. A method of treating an abnormal spine segment of a patient, comprising:
implanting a stabilization device such that a first superior end of the device engages a first spinous process, and such that a second inferior end of the device engages a second non-adjacent spinous process to thereby off-load at least one level of intervertebral discs and facet joints between said first and second engaged spinous processes.

24. The method of claim 23, wherein the stabilization device off-loads two levels of intervertebral discs and facet joints between the first and second engaged spinous processes.

25. The method of claim 23, wherein the stabilization device off-loads three levels of intervertebral discs and facet joints between the first and second engaged spinous processes.

26. The method of claim 23, wherein the stabilization device off-loads at least four levels of intervertebral discs and facet joints.

27. The method of claim 23, further comprising coupling a medial portion of the stabilization device to a pedicle fixated member.

28. The method of claim 27, wherein coupling the medial portion of the stabilization device comprises slidably coupling the medial portion to the pedicle fixated member.

29. The method of claim 23, wherein the first superior end and the second inferior end grippingly engage the non-adjacent spinous processes.

30. The method of claim 23, further comprising adjusting a length of a medial portion between the first superior end and the second inferior end.

31. The method of claim 23, wherein the stabilization device limits extension of the spine segment.

32. The method of claim 23, wherein the stabilization device limits flexion of the spine segment.
